(19) United States
(12) Patent Application Publication
(10) Pub. No.: US 2025/0297230 A1
(43) Pub. Date: Sep. 25, 2025

(54) METHOD FOR PREPARING EXTRACELLULAR MATRIX-INDUCED SELF-ASSEMBLY-BASED 3D PRINTED ARTIFICIAL TISSUE, AND ARTIFICIAL TISSUE PREPARED THEREBY

(71) Applicant: AJOU UNIVERSITY INDUSTRY-ACADEMIC COOPERATION FOUNDATION, Suwon-si, Gyeonggi-do (KR)

(72) Inventors: Doyoung PARK, Suwon-si (KR); Heewoong YUN, Suwon-si (KR); Sujin NOH, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 18/860,204

(22) PCT Filed: Apr. 26, 2023
(86) PCT No.: PCT/KR2023/005678

§ 371 Date: Oct. 25, 2024

(30) Foreign Application Priority Data
Apr. 28, 2022 (KR) 10-2022-0052962

(51) Int. Cl. C12N 5/071; B33Y 10/00; B33Y 40/10; B33Y 70/00; B33Y 80/00; C12N 5/0775

(52) U.S. Cl. CPC C12N 5/0697; B33Y 40/10; B33Y 70/00; B33Y 80/00; C12N 5/0662; B33Y 10/00; C12N 2513/00; C12N 2533/90

(57) ABSTRACT

The present invention relates to a method for preparing an extracellular matrix-induced self-assembly-based 3D printed artificial tissue, and artificial tissue prepared thereby, and provides: a method in which a self-assembly, formed by inducing stem cell differentiation using extracellular matrix-derived biomaterials, is applied to 3D printing so that artificial tissue can be fine-patterned with widths in units of micrometers and morphological appearance of origin tissue can be implemented; and artificial tissue printed in a mature tissue form, which is not that of a cell-biomaterial mixture from the time of printing. The artificial tissue prepared by the preparation method of the present invention mimics the biological characteristics of a target organ according to the origin of the extracellular matrix, and thus enables artificial tissue and artificial organs very similar to actual original tissue to be provided.

### TECHNICAL FIELD

The present invention relates to a method for preparing an extracellular matrix (ECM)-induced self-assembly-based 3D printed artificial tissue, and artificial tissue prepared thereby, and provides a method for preparing an artificial tissue, in which a self-assembly formed by inducing stem cell differentiation using ECM-derived biomaterials is applied to 3D printing so that artificial tissue can be finely patterned with widths in the micrometer range and implement the morphological appearance of the originating tissue; and the resulting artificial tissue printed in the form of mature tissue rather than a mixture of cells and biomaterials from the time of printing. The artificial tissue prepared by the preparation method of the present invention mimics the biological characteristics of a target organ according to the origin of the ECM, and thus enables artificial tissue and artificial organs very similar to actual original tissue to be provided.

### BACKGROUND ART

Recently, in the field of tissue engineering, the technology of extracting homologous or heterologous organs and tissue, removing cells (decellularization), and using the remaining organs and tissue as various types of tissue engineering preparations has been attracting attention. To date, various tissue-derived biomaterials such as small intestinal submucosa, bladder, skin, amniotic membrane, bone, ligament, and cartilage have been commercialized or are being studied. The methods of producing tissue-engineered artificial organs using tissue-derived biomaterials reported so far include salt leaching, electrospinning, and 3D printing.

Among them, 3D printing is being studied extensively because it can utilize various types of materials and cells and implement the desired shapes. In particular, liquefying the above-mentioned tissue-derived biomaterials or mixing them in powder form and printing them together with cells increases cell activity, and the possibility of inducing the biomaterials into tissue, such as the expression of specific genes and proteins of the originating tissue, has been confirmed. However, there are still limitations in terms of tissue induction and maturity, such as using synthetic materials that may generate byproducts harmful to cells when decomposed or forming non-uniform cell-biomaterial complexes.

For example, Korean Patent Publication No. 10-2020-0066218 discloses a technology for preparing a bioink composition containing microparticles of human tissue and a structure using the same, but the technology requires separate cell growth factors and differentiation factors to control cell functions and differentiation, and a structure manufactured through 3D printing must undergo a cross-linking step to satisfy bioink printability and mechanical properties after printing.

Against this backdrop, the present inventors have attempted to provide a method for preparing artificial tissue that is improved over the related art by providing a method for printing self-assembled tissue that is tissue-specifically differentiated through self-assembly using cells and extracellular matrix-derived biomaterials without using differentiation factors and mature at the time of printing.

### DISCLOSURE

### Technical Problem

Therefore, an object of the present invention is to provide a technology for preparing artificial tissue having biochemical characteristics similar to those of the originating tissue by applying a self-assembled cell-biomaterial complex (cell-decellularized extracellular matrix (DECM) self-assembly) with excellent capacity of inducing differentiation and maturation into tissues for printing.

Specifically, an object of the present invention is to provide a method for preparing cell-DECM self-assembly-based 3D printed artificial tissue.

Another object of the present invention is to provide cell-DECM self-assembly-based 3D printed artificial tissue and artificial organs prepared by the above-described method.

### Technical Solution

To solve the above-described technical problem, the present invention provides a method for preparing cell-decellularized extracellular matrix (DECM) self-assembly-based 3D printed artificial tissue, including:
(a) decellularizing and powdering a tissue-derived extracellular matrix (ECM) to prepare DECM powder;
(b) forming a cell-DECM self-assembly by adding the DECM powder to a culture medium containing cells and then culturing the same;
(c) preparing a tissue strand ink by homogenizing the cell-DECM self-assembly; and
(d) preparing 3D printed artificial tissue by applying the homogenized tissue strand ink to a 3D printing device.

According to one preferred embodiment of the present invention, the tissue of Step (a) may be bone, ligament, muscle, fibrocartilage, or cartilage.

According to another preferred embodiment of the present invention, the cells in Step (b) may be stem cells.

According to still another preferred embodiment of the present invention, the stem cells may be one or more selected from the group consisting of mesenchymal stem cells, embryonic stem cells, and induced pluripotent stem cells.

According to yet another preferred embodiment of the present invention, the DECM powder in Step (b) may be added at a concentration of 0.05 to 3 mg/ml.

According to yet another preferred embodiment of the present invention, the cell-DECM self-assembly in Step (b) may be formed in vitro.

According to yet another preferred embodiment of the present invention, the cell-DECM powder self-assembly in Step (b) may be formed by inducing cell proliferation or cell differentiation.

According to yet another preferred embodiment of the present invention, the method may further include adding a solubilized DECM solution in Step (b).

According to yet another preferred embodiment of the present invention, the solubilized DECM solution may be added at a concentration of 50 to 500 μg/ml.

According to yet another preferred embodiment of the present invention, Step (b) may be culturing for two to nine days after the cells and the DECM powder begin to fuse.

According to yet another preferred embodiment of the present invention, the homogenizing of the cell-DECM self-assembly in Step (c) may be blending by passing the cell-DECM self-assembly obtained after Step (b) through a molecular sieve or through a syringe connector connected to a nozzle.

According to yet another preferred embodiment of the present invention, the mesh diameter of the molecular sieve may be 50 to 800 μm, and the diameter of the nozzle connected to the syringe connector may be 1 to 3 mm.

According to yet another preferred embodiment of the present invention, the tissue strand ink prepared in Step (d) may be injected into a syringe for 3D printing to perform 3D printing with a nozzle size of 200 μm or more under an air pressure of 20 to less than 150 kPa at a printing speed of 0.1 to 3 mm/sec.

In addition, the present invention provides cell-decellularized extracellular matrix (DECM) self-assembly-based 3D printed artificial tissue and artificial organs prepared by the above-described method.

According to one preferred embodiment of the present invention, the artificial tissue and the artificial organs exhibit biochemical characteristics of original tissue.

### Advantageous Effects

A method for preparing 3D printed artificial tissue of the present invention enables fine patterning with widths in the micrometer range, and it allows not only the implementation of the morphological appearance of the original tissue, but also the maturation into tissue that mimics the biological characteristics of the target organ depending on the origin of the extracellular matrix. In addition, unlike existing methods, it is possible to print in the form of tissue that is a mature self-assembly rather than a combination of cells and biomaterials at the time of printing. Accordingly, the artificial tissue prepared by the method of the present invention can be utilized in the development of medical products required for regenerative medicine, for example, bone, ligament, muscle, cartilage, or meniscus damage. In addition, since the method can be used to produce a tissue engineering product suitable for the anatomical location, characteristics, and physicochemical requirements of a target tissue, a wide range of applications can be expected.

### DESCRIPTION OF DRAWINGS

FIGS. 1A and 1B illustrate the results of visual analysis and biochemical analysis before and after the decellularization process of each tissue (bone, ligament, muscle, fibrocartilage, and cartilage tissue). Specifically, FIG. TA illustrates the results of visual analysis of the parent tissue before the decellularization process (Native) and the tissue after the decellularization process (Decelled), and FIG. 1B illustrates the results of biochemical substance content analysis of the parent tissue before the decellularization process (Native) and the tissue after the decellularization process (Decelled).

FIGS. 2A to 2D illustrate the results of preparing cell/decellularized extracellular matrix (DECM) self-assemblies. Specifically, FIG. 2A shows a photograph of a high-density culture of porcine synovial membrane-derived stem cells and a photograph after DECM treatment, FIG. 2B shows photographs illustrating the condensation of cell/DECM self-assemblies, FIG. 2C shows the results of visual analysis and live/dead assay according to the ECM concentration of cell/DECM self-assemblies, and FIG. 2D shows the results of quantitative analysis based on live/dead assay.

FIGS. 3A and 3B illustrate the results of enhancing the cartilage tissue differentiation capacity of self-assemblies by treatment with solubilized cartilage DECM. Specifically, FIG. 3A shows graphs illustrating the results of analyzing the increase and decrease in cartilage-related genes in self-assemblies by treatment with solubilized cartilage DECM, and FIG. 3B shows photographs illustrating the results of histological analysis of self-assemblies by treatment with solubilized cartilage DECM.

FIGS. 4A to 4C illustrate the results of preparing a tissue strand ink through a homogenization process of cell/DECM self-assemblies. FIG. 4A shows a graph comparing the 3D printing structure shape and cell viability of the tissue strand ink according to the culture period of the self-assembly, FIG. 4B shows graphs comparing the printability of the tissue strand ink according to the mesh diameter of a molecular sieve used in the homogenization process of the self-assembly, and FIG. 4C shows graphs comparing the printability of the tissue strand ink according to the diameter of a syringe nozzle used in the homogenization process of the self-assembly.

FIGS. 5A and 5B illustrate the characterization results of the artificial tissue printed with the cell/DECM self-assembly-based tissue strand ink. Specifically, FIG. 5A shows photographs illustrating the process of loading the cell/DECM self-assembly-based tissue strand ink and preparing artificial tissue using 3D printing, and FIG. 5B shows a graph comparing the cell viability of the artificial tissue using 3D printing compared to the cell/DECM self-assembly-based tissue strand ink.

FIGS. 6A to 6D illustrate the results of biochemical characterization of the artificial tissue printed with the cell/DECM self-assembly-based tissue strand ink according to the use of tissue-specific DECM. Specifically, FIG. 6A shows the result of visual observation of the artificial tissue printed with the tissue-specific cell/DECM self-assembly-based tissue strand ink, FIG. 6B shows the results of protein profile analysis of the artificial tissue printed with the tissue-specific cell/DECM self-assembly-based tissue strand ink, FIG. 6C shows the results of collagen analysis of the artificial tissue printed with the tissue-specific cell/DECM self-assembly-based tissue strand ink, and FIG. 6D shows the results of sulfated glycosaminoglycan (sGAG) analysis of the artificial tissue printed with the tissue-specific cell/DECM self-assembly-based tissue strand ink.

FIGS. 7A to 7E illustrate the results of evaluating the degree of tissue differentiation of the artificial tissue printed with the tissue-specific cell/DECM self-assembly-based tissue strand ink and show the results of evaluating the degree of tissue differentiation of artificial tissues printed with cartilage (FIG. 7A), fibrocartilage (FIG. 7B), bone (FIG. 7C), ligament (FIG. 7D), and muscle (FIG. 7E) tissue strand inks, respectively.

FIG. 8 shows a schematic diagram illustrating a method for preparing cell-DECM self-assembly-based 3D printed artificial tissue of the present invention.

### MODES OF THE INVENTION

Hereinafter, the present invention will be described in more detail.

Meanwhile, each description and embodiment disclosed in the present application may also be applied to the other descriptions and embodiments. In other words, all combinations of various elements disclosed in the present application fall within the scope of the present invention. In addition, the scope of the present invention is not limited by the specific description described below.

In addition, those skilled in the art may recognize or confirm many equivalents to the specific embodiments of the present invention described in the present application using only common experiments. In addition, such equivalents are intended to be included in the present invention.

As described above, 3D printing is being studied extensively because various materials and cells may be utilized and a desired shape may be implemented. However, there are still limitations in terms of tissue induction and maturity, such as using synthetic materials that may generate byproducts harmful to cells when decomposed or forming non-uniform cell-biomaterial complexes. Accordingly, the present inventors have found a solution to the above-described problems by forming a self-assembly with a homogeneous distribution of cells and extracellular matrix (ECM) without using separate synthetic materials or growth factors and by applying this to 3D printing technology to derive an optimized 3D printing method. The method of preparing 3D printed artificial tissue of the present invention enables fine patterning with widths in the micrometer range, and it allows not only the implementation of the morphological appearance of the original tissue, but also the maturation into tissue that mimics the biological characteristics of the target organ depending on the origin of the ECM.

Therefore, a first aspect of the present invention relates to a method for preparing cell-decellularized extracellular matrix (DECM) self-assembly-based 3D printed artificial tissue.

Specifically, the preparation method includes the following steps:
(a) decellularizing and powdering a tissue-derived ECM to prepare DECM powder;
(b) forming a cell-DECM self-assembly by adding the DECM powder to a culture medium containing cells and then culturing the same;
(c) preparing a tissue strand ink by homogenizing the cell-DECM self-assembly; and
(d) preparing 3D printed artificial tissue by applying the homogenized tissue strand ink to a 3D printing device.

In the preparation method of the present invention, Step (a) is a step of preparing DECM powder, wherein the decellularization is performed to eliminate an immune response to the cellular components of a heterologous tissue. In order to achieve effective decellularization, the cellular components of the tissue must be completely eliminated, and while maintaining the physical properties of the tissue, the biochemical characteristics must be preserved to the greatest extent possible so that the ECM may be used as a tissue support in the field of tissue engineering, and various cleaning agents and chemicals used in the processing must be completely eliminated.

For the decellularization process of Step (a), any method known in the art may be applied without limitation. For example, a part of the desired tissue may be obtained from an animal or human tissue or organ, washed, freeze-dried, and freeze-crushed to prepare powder, and then the prepared powder may be dissolved in a hypotonic solution for a certain period of time and treated with a solution containing a surfactant to perform decellularization. Alternatively, a tissue-derived ECM may be first decellularized and then powdered.

The tissue-derived ECM may be derived from artificial tissue or an artificial organ to be ultimately prepared, and may be derived from, for example, fat, muscle, cartilage, fibrocartilage, the heart, bone, ligament, skin, blood vessels, the lungs, the comeas, the brain, mucosal epithelial tissue, the bladder, the liver, the kidneys, the esophagus, the testes, the uterus, the placenta, nerves, the spinal cord, the pancreas, the spleen, the intestines, and the like, but is not limited thereto.

As the surfactant, an anionic surfactant, for example, sodium dodecyl sulfate (SDS), and a nonionic surfactant, for example, Triton X-100, may be used, but the present invention is not limited thereto. A preferred concentration of SDS is 0.10% to 0.5%, and a preferred concentration of Triton X-100 is 0.5% to 1%. The hypotonic solution is used together with a surfactant to increase the decellularization efficiency. A preferred hypotonic solution is, for example, 5 to 10 mM Tris-HCl (pH 7.4), but is not limited thereto.

The decellularization may be performed by treating the tissue powder in a hypotonic solution for two to six hours and then treating it in a solution containing a surfactant for one to four hours, and this process is performed at 4° C. to room temperature (e.g., 4 to 35° C.).

Finally, to eliminate the genetic material present in the tissue powder, it is treated with a DNAase and stirred for 10 to 12 hours.

After eliminating the genetic material, the DECM powder is finally prepared through freeze-drying, and the powder may be prepared as fine powder with a particle size of 25 to 100 μm or less. When fine particles larger than the above range are used, the biological and physical characteristics of the cells may change, and ultimately, differentiation control may be affected. In addition, when fine particles smaller than the above range are used, the yield is low and additional time is required due to limitations in the internal preparation process, so their use is limited.

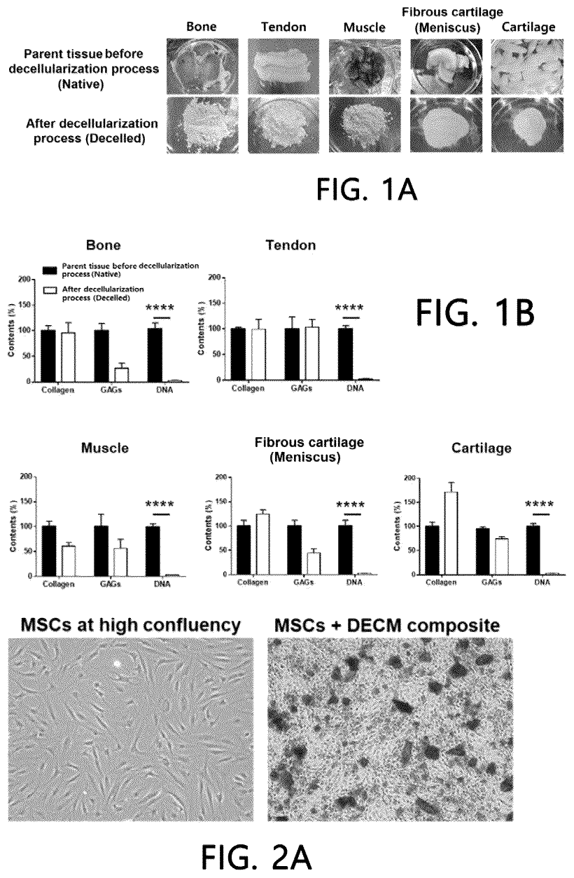

In one specific embodiment of the present invention, collagen, sulfated glycosaminoglycan (sGAG), and DNA contents of the DECM powder prepared through the process were analyzed. As a result, as shown in FIG. 1B, the collagen and sGAG contents of the tissue were well maintained even after decellularization, and 97% or more of the DNA was removed, confirming that the decellularization was successfully performed.

In the preparation method of the present invention, Step (b) is a step of forming a cell-DECM self-assembly, which is performed by adding the DECM powder prepared in Step (a) to a culture medium containing cells and then culturing the cells.

In Step (b), the cells may be stem cells, which may be autologous or xenogeneic stem cells, and specifically may be one or more selected from the group consisting of mesenchymal stem cells, embryonic stem cells, and induced pluripotent stem cells, but are not limited thereto. The cells are preferably seeded at 1.5×10 to 4×10 cells and cultured until the culture rate reaches 90% or higher. When a smaller number of cells than the above range are used, the accumulation of ECM may be limited, and self-assembly may not proceed. In addition, when a greater number of cells than the above range are used, the supply of oxygen and nutrients to some cells may not be smooth, which may affect the viability of the cells.

Thereafter, the DECM powder prepared in Step (a) may be added to the culture medium and cultured for a certain period of time. At this time, the DECM powder may be added at a concentration of 0.05 to 3 mg/ml, preferably 1 to 2.5 mg/ml, but is not limited thereto. The culture time may be up to 48 hours, preferably 12 to 24 hours.

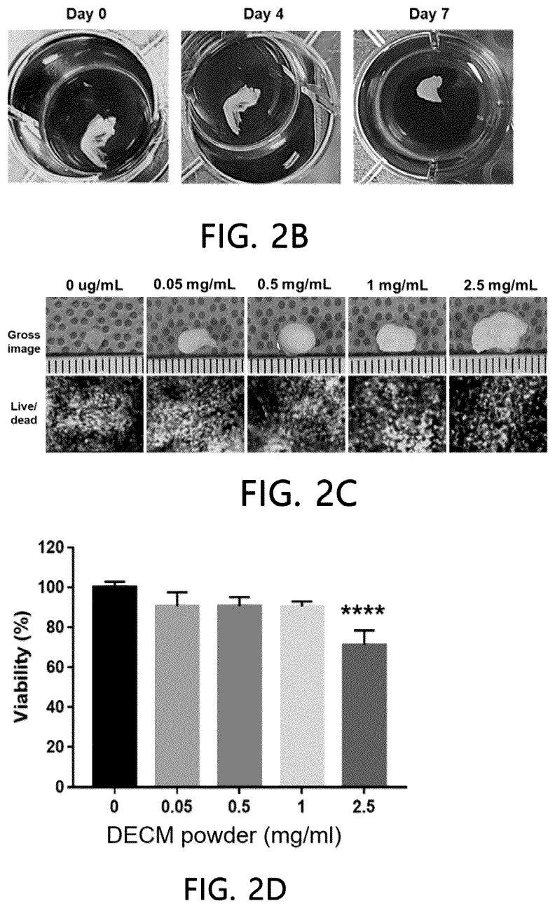

In one specific embodiment of the present invention, the cell viability of the cell-DECM self-assembly-based tissue strand ink according to the concentration of the DECM powder was analyzed to establish the optimized concentration of the DECM powder. As a result of using the chondrogenic ECM powder in the concentration range of 0 to 2.5 mg/ml, as shown in FIGS. 2C and 2D, when treated at 2.5 mg/ml, the cell viability was approximately 72%, and significant cell death was observed compared to the control group and the groups treated with other concentrations of the ECM powder.

In Step (b), the DECM powder may not only act as a chemoattractant that attracts cells, but also has a strong binding ability to cells and the ability to promote proliferation and differentiation. Since the DECM induces differentiation depending on the type of tissue from which it is derived, it can produce various biomimetic structures. Therefore, the DECM powder is effective in cell attachment and proliferation, and in particular, may have a great impact on the differentiation of stem cells into specific cells.

The cell-DECM self-assembly in Step (b) may be formed in vitro.

In addition, in Step (b), a cell-DECM self-assembly may be formed by inducing cell proliferation or cell differentiation. After the cells and the DECM begin to fuse, when additional culture is performed for a certain period of time, a cell-DECM self-assembly that gradually condenses through self-assembly is produced.

In order to enhance the tissue differentiation ability of the cell-DECM self-assembly in Step (b), a solubilized DECM solution may be further added. The solubilized DECM solution may be prepared, for example, by stirring DECM powder with pepsin in a 0.01 M to 0.5 M hydrochloric acid aqueous solution or a 0.1 M to 0.5 M acetic acid aqueous solution at 4° C. to 36° C., and neutralizing the pH using a NaOH solution. For the solubilized DECM solution, a dialysis membrane (MWCO: 1,000 to 3,000 Da) may be used to eliminate a salt (NaCl) generated during the neutralization process, and the pH, ion concentration, and osmotic pressure may be adjusted by adding a phosphate buffer solution (PBS). In addition, the ECM derived from the same tissue as the DECM powder prepared in Step (a) may be added at a concentration of 50 to 500 μg/ml, but is not limited thereto. The solubilized DECM solution may preferably be added at the time of forming the self-assembly and at each time the culture medium in the self-assembly is replaced.

In the preparation method of the present invention, Step (c) is a step of preparing a tissue strand ink for use by applying the cell-DECM self-assembly obtained in Step (b) to a 3D printing device, and a tissue strand ink optimized for use in 3D printing is prepared through a homogenization process.

In the present invention, the term “tissue strand ink” refers to a 3D tissue culture obtained by homogeneously blending a heterogeneous self-assembly.

In the preparation method of the present invention, Step (c), that is, the process of preparing a tissue strand ink from a self-assembly, includes a process of homogeneously blending an initial immature self-assembly having physical properties suitable for printing.

The self-assembly obtained in Step (b) has limitations in direct application to 3D printing because it is physically/biochemically heterogeneous and has poor printability.

Accordingly, in one specific embodiment of the present invention, a tissue strand ink was prepared by adjusting the culture period and/or blending process of the cell-DECM self-assembly, and its printability and cell viability were confirmed.

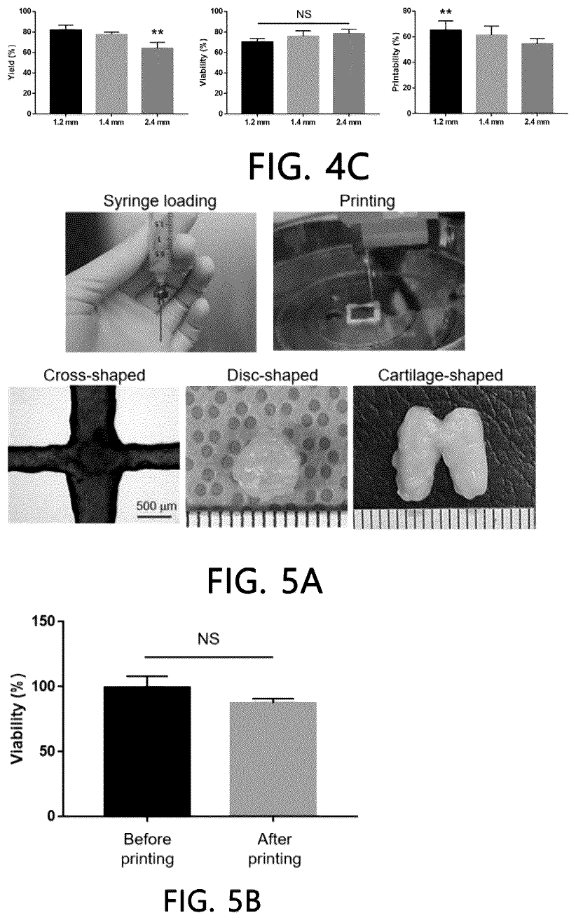

First, the printability and cell viability of the tissue strand ink prepared through the blending process were confirmed after culturing for 1, 3, 7, and 10 days, respectively, after the cells and the DECM began to fuse. As a result, as shown in FIG. 4A, when the culture period of the self-assembly was less than 3 days, proper fusion between the cells and the ECM did not occur, resulting in poor adhesiveness and difficulty in forming a 3D structure. In addition, when the culture period exceeded 10 days, the bonding between the cells and the ECM was too strong, making it difficult to achieve physical homogeneity of the ink, and the physical stress increased during the blending process, resulting in significant cell death.

Therefore, the optimal culture period of the self-assembly for use as a tissue strand ink may be two to nine days, more preferably three to eight days, and most preferably three to seven days after the cells and the DECM begin to fuse.

In addition, the yield, cell viability, and printability of the tissue strand ink according to the blending process of the cultured self-assembly were confirmed. The blending process of the self-assembly for producing a homogeneous tissue strand ink may be performed, for example, by passing the cultured self-assembly through a molecular sieve or a syringe nozzle.

First, blending was performed using molecular sieves having various mesh diameters of 50 to 800 μm (50, 100, 200, 400, and 800 μm, respectively), and the yield, cell viability, and printability of the tissue strand ink according to the mesh diameter were confirmed. As a result, as shown in FIG. 4B, as the mesh diameter increased, the yield and cell viability of the tissue strand ink increased, and the print resolution (printability) decreased. Therefore, when the yield, cell viability, and printability of the tissue strand ink are comprehensively considered, it is appropriate to perform blending of the self-assembly using a molecular sieve having a mesh diameter of 50 to 800 μm, more preferably 100 to 600 μm, and most preferably 200 to 400 μm, but the mesh dimeter is not limited thereto. The blending process using the molecular sieve may be performed repeatedly several times until the particle size of the tissue strand ink becomes homogenous, and for example, it may be performed repeatedly one to five times, but is not limited thereto.

Second, the cultured self-assembly was repeatedly passed through syringe nozzles having various diameters from 1.2 mm to 2.4 mm (1.2, 1.4, and 2.4 mm, respectively) to perform blending, and then the yield, cell viability, and printability of the tissue strand ink were confirmed. As a result, as shown in FIG. 4C, as the diameter of the syringe nozzle decreased, the yield and printability were improved, and the cell viability was at a constant level regardless of the diameter of the syringe nozzle. Therefore, when the yield, cell viability, and printability of the tissue strand ink are comprehensively considered, it is appropriate to perform blending of the self-assembly using a syringe having a nozzle diameter of 1.0 to 3.0 mm, more preferably 1.0 to 2.7 mm, and most preferably 1.2 to 2.4 mm, but the nozzle diameter is not limited thereto. The blending process using the molecular sieve may be performed repeatedly several times until the particle size of the tissue strand ink becomes homogenous, and for example, it may be performed repeatedly one to five times, but is not limited thereto.

In the preparation method of the present invention, Step (d) is a step of applying the homogenized tissue strand ink from Step (c) to a 3D printing device to prepare 3D printed artificial tissue. Since the cell-DECM self-assembly-based tissue strand ink applied to a 3D printing device in Step (d) contains living cells and ECM, it is preferable to perform 3D printing under conditions for maximizing cell viability. For example, the homogenized tissue strand ink obtained in Step (c) is preferably injected into a 3D printing syringe to perform 3D printing with a nozzle size of 200 Vim or more under an air pressure of 20 to less than 150 kPa at a printing speed of 0.1 to 3 mm/sec.

In one specific embodiment of the present invention, the shape of artificial tissue prepared by performing 3D printing under the above conditions was observed and the cell viability thereof was confirmed. As a result, as shown in FIG. 5A, not only a fine cross-shaped structure of 500 μm in size but also a large structure of 1 cm or larger could be prepared, and as shown in FIG. 5B, the artificial tissue had a cell viability of 85% or more compared to the tissue strand ink even after printing.

Accordingly, the method for preparing cell-DECM self-assembly-based 3D printed artificial tissue of the present invention is able to prepare a tissue strand ink having conditions optimized for use in 3D printing through the above-described homogenization process, and by applying this to 3D printing, it is possible to obtain artificial tissue and artificial organs finely patterned with widths in the micrometer range (e.g., 200 to 700 μm).

Therefore, a second aspect of the present invention relates to cell-DECM self-assembly-based 3D printed artificial tissue and artificial organs prepared by the above-described method.

The cell-DECM self-assembly-based 3D printed artificial tissue and artificial organs prepared by the above-described method allows the implementation of the morphological appearance of the original tissue and the maturation into tissue that mimics the biological characteristics of the target organ depending on the origin of the ECM.

Hereinafter, the present invention will be described in more detail by the following examples. However, the following examples are only intended to illustrate the present invention, and the scope of the present invention is not limited thereto.

### MODE FOR INVENTION

### Example 1

Preparation of Decellularized Extracellular Matrix (DECM) Powder Derived from Bone, Ligament, Muscle, Fibrocartilage, and Cartilage Tissue

Porcine bones were harvested from the tibia and femoral condyle, ligaments (tendons) from the patellar tendon, muscles from the quadriceps, and fibrocartilage and cartilage tissue from the knee using a surgical blade and a saw, respectively. Each tissue sample was washed three times with distilled water and then powdered through freeze-drying and freeze-crushing.

The obtained tissue powder was added to a hypotonic solution of 10 mM Tris-HCl at pH 7.4 and kept at room temperature for four hours, and then the resulting mixture was treated with a Tris-buffered saline (TBS) buffer containing 0.1% sodium dodecyl sulfate (SDS) for two hours to perform decellularization. Thereafter, the DECM powder was washed six times with distilled water to remove the surfactant component SDS. Finally, a solution containing DNAase was added to remove the genetic material present in the tissue ECM powder, and the resulting mixture was stirred for 12 hours. Thereafter, the powder was washed with distilled water six times to complete the decellularization process.

Finally, as shown in FIG. TA, the resulting powder was freeze-dried to prepare decellularized extracellular matrix (DECM) powder, which was then sieved to prepare fine powder with a particle diameter of 100 μm or less.

### Example 2

Analysis of Biochemical Characteristics of DECM Powder Derived from Bone, Ligament, Muscle, Fibrocartilage, and Cartilage Tissue
2-1. Analysis of Collagen and Sulfated Glycosaminoglycan (sGAG) Contents in Decm Powder Derived from Decellularized Bone, Ligament, Muscle, Fibrocartilage, and Cartilage Tissue

To quantitatively analyze the collagen and sGAG components maintained after the decellularization process, the Sircol collagen assay and the Blyscan sGAG assay were performed.

As a result, as shown in FIG. 1B, it was confirmed that the collagen content was well maintained after decellularization in the four types of tissue except for the muscle tissue, and in particular, it was confirmed that the collagen content increased after decellularization in the fibrocartilage and cartilage tissue.

In the case of sGAG content, more than 50% of the native tissue was maintained even after the decellularization process in the four types of tissue except for the bone tissue, and in particular, the ligament tissue showed no statistical significance even after the decellularization process compared to the native tissue.

2-2. Analysis of DNA Content in DECM Powder Derived from Decellularized Bone, Ligament, Muscle, Fibrocartilage, and Cartilage Tissue

The double stranded DNA (dsDNA) content of the ECM powder of bone, ligament, muscle, fibrocartilage, and cartilage tissue after the decellularization process was analyzed using the PicoGreen assay.

As a result, as shown in FIG. 1B, 97% or more of DNA was removed from the five types of tissue, and the absolute amount was 50 ng or less per 1 mg of tissue. Therefore, it was confirmed that the decellularization was successfully performed on the five types of tissue.

From the results, it was confirmed that the decellularization process performed on the five types of tissue efficiently reduced genetic material while maintaining major substances such as sGAG and collagen.

### Example 3

### Preparation of Stem Cell/DECM Self-Assembly for 3D Printing

### 3-1. Preparation of Cell/DECM Self-Assembly

Cell/DECM self-assemblies using the DECM powder from each type of tissue were prepared through the following process.

As shown in FIG. 2A, porcine synovial mesenchymal stem cells (pSYMSCs) were seeded at 2.5×10 cells in a culture dish with a diameter of 60 mm and cultured in an incubator for up to 48 hours to achieve a confluency of 90% or more. Thereafter, each prepared tissue-derived DECM powder was suspended in a cell culture medium at a concentration of 1 mg/ml and cultured for up to 48 hours. When the stem cells and the DECM began to fuse, they were separated from the culture dish using a cell scraper and transferred to a 6-well plate, to which 5 ml of the culture solution was added, and the cell culture solution was replaced with a fresh cell culture solution every three days.

As shown in FIG. 2B, it was confirmed that stem cell/DECM self-assemblies in a gradually condensed form were prepared through self-assembly over a period of one week after stem cell/DECM fusion.

### 3-2. Optimization of DECM Concentration in Cell/DECM Self-Assembly for 3D Printing

To analyze the cell viability of the stem cell/DECM self-assembly-based tissue ink according to the concentration of ECM, 0 to 2.5 mg/ml of cartilage ECM powder was suspended in the cell culture solution to prepare self-assemblies, and a live/dead assay was performed on day 7 of culture.

As a result, as shown in FIGS. 2C and 2D, it was confirmed that, as the concentration of the DECM powder increased, the volume of the self-assembly increased. Meanwhile, when the self-assembly was treated with the DECM powder at a concentration of 2.5 mg/ml or higher, cell viability was approximately 72%, and significant cell death was observed compared to the control group and the DECM treatment groups at other concentrations.

### Example 4

Enhancement of Tissue Differentiation Capacity of Cell/DECM Self-Assemblies by Treatment with Solubilized Cartilage DECM

To analyze whether the tissue differentiation capacity of cell/DECM self-assemblies is enhanced by treatment with solubilized DECM (DECM-Sol), a reverse transcription quantitative polymerase chain reaction (RT-qPCR) and a histological analysis were performed on day 14 of self-assembly culture. To this end, DECM-Sol was treated at a concentration of 250 μg/ml each time the culture solution was replaced for two weeks from the day when stem cells/DECM started to form a self-assembly.

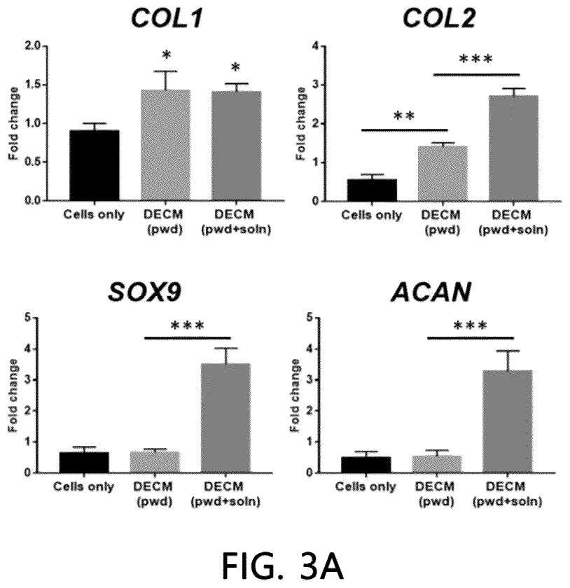

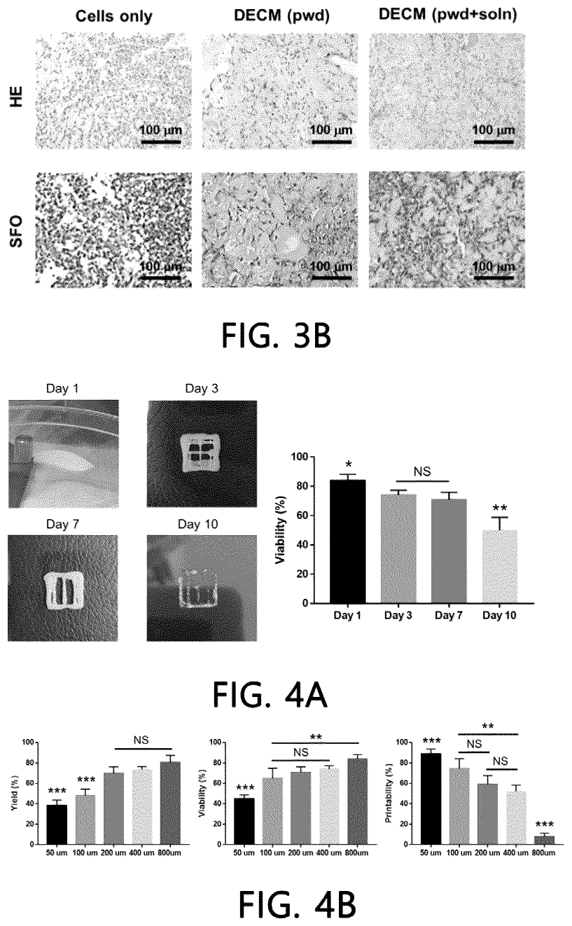

As a result, as shown in FIG. 3A, the self-assembly group further treated with DECM-Sol exhibited a significant increase in the expression of cartilage-specific markers (COL2, SOX9, and ACAN) compared to the stem cell-only group and stem cell/DECM self-assembly group.

In addition, as a result of hematoxylin and eosin (H&E) staining of the self-assembly, as shown in FIG. 3B, it was confirmed that the distribution of cells and ECM was most homogeneous in the group treated with the DECM powder+DECM-Sol compared to the stem cell-only group and the group treated with DECM powder only. In the safranin-O staining analysis, it was confirmed that the expression of sGAG was significantly enhanced in the stem cell/DECM self-assembly prepared by adding DECM-Sol compared to the other two groups.

### Example 5

### Optimization of Homogenization Process of Cell/DECM Self-Assembly-Based Tissue Strand Ink

The process of preparing a tissue strand ink from self-assemblies includes a process of homogeneously blending initial immature self-assemblies that have physical properties suitable for printing and have been cultured for one to ten days. At this time, as the self-assembly culture period increases, cells and ECM become more aggregated, so the adhesiveness and physical rigidity of the self-assembly may increase, and the cell viability in the tissue strand ink may decrease due to the stress generated during the blending process. Therefore, in this example, in order to optimize the homogenization operation of cell/DECM self-assembly-based tissue strand ink, the yield, cell viability, and printability of the tissue strand ink were evaluated by adjusting the self-assembly culture period and blending process.

### 5-1. Self-Assembly Culture Period Optimization

Tissue strand inks were prepared by culturing cells and DECM for 1, 3, 7, and 10 days, respectively, a blending process was performed after the cells and DECM began to fuse, and the printability and cell viability of the tissue strand inks were confirmed. The results are shown in FIG. 4A.

As shown in the left photograph of FIG. 4A, it was confirmed that the tissue strand ink prepared with the self-assembly on day 1 of culture had weak adhesiveness due to insufficient aggregation between the cells and the ECM, making it difficult to maintain its shape during printing, and the structure easily collapsed. Meanwhile, the tissue strand inks prepared respectively with the self-assemblies on day 3 and day 7 of culture exhibited improved printing resolution compared to the day 1 group, and fabrication of a stable 3D structure was possible. However, the tissue strand ink prepared with the self-assembly on day 10 of culture had high physical rigidity due to high aggregation between the cells and the ECM, and ink homogeneity was poor and the ink was not suitable for printing even after the blending process.

In the case of cell viability, the cell viability after preparing the tissue strand ink through the blending process was evaluated in comparison with that before the process. In order to observe the change in cell viability before and after the blending process, the self-assembly and the tissue strand ink after the blending process were prepared with the same weight, treated with collagenase for four hours and separated into single cells, and then treated with a trypan blue solution to measure the number of viable cells using a cell counter. At this time, the cell viability is a value expressed as a percentage calculated with reference to the viability of the self-assembly before the blending process as 100%. As a result, as shown in the right graph of FIG. 4A, the cell viability was the highest at 85.3% in the group on day 1 of culture, and tended to decrease as the culture period increased. The groups on day 3 and day 7 of culture both showed a viability of 70% or more, and there was no statistical difference between the two groups. Finally, the group on day 10 of culture exhibited the lowest cell viability at 49.8%.

Through the above results, it can be seen that the optimal culture period for self-assemblies for use as tissue strand inks is two to nine days after the cells and the DECM begin to fuse.

### 5-2. Optimization of Blending Process of Cultured Self-Assembly

The self-assembly blending process may be performed by repeatedly passing the self-assembly through a molecular sieve having a mesh diameter in the micrometer range, or through a syringe connector connected to a nozzle having a diameter in the millimeter range.

Therefore, to optimize the self-assembly blending process, the cultured self-assembly was passed through a molecular sieve or syringe nozzle, and then the yield, cell viability, and printability of the tissue strand ink were evaluated.

First, in the case of the blending process using a molecular sieve, the self-assembly was placed on a sterilized sieve and then moved left and right using a cell scraper to pass it through the sieve. At this time, molecular sieves with mesh diameters of 50, 100, 200, 400, and 800 μm were used, respectively, and the yield, cell viability, and printability of the tissue strand ink according to the mesh diameter were confirmed. The yield of the tissue strand ink was evaluated by calculating the weight of the tissue strand ink obtained after the blending process as a percentage with reference to 1 g of the self-assembly as 100%. As shown in the left graph of FIG. 4B, the yield of the tissue strand ink tended to decrease as the mesh diameter of the molecular sieve decreased. In particular, the yield was significantly decreased at mesh diameters of 100 μm or less, and there was no significant difference at mesh diameters of 200 μm or more (50 μm: 38.1%, 100 μm: 48.2%, 200 μm: 69.7%, 400 μm: 73.5%, 800 μm: 80.5%).

Cell viability was confirmed in the same manner as in Example 4-1. As shown in the middle graph of FIG. 4B, cell viability tended to increase as the mesh diameter of the molecular sieve increased. When a mesh diameter of 50 μm was used, the cell viability was approximately 44.7%, indicating significant cell death, but a cell viability of approximately 70% or more was observed for diameters of 100 μm or more, and a cell viability of approximately 83.8% was observed in the 800 μm group.

With regard to the printability, printing precision was calculated as a percentage by linearly printing a 4×4 mm square structure and then analyzing the area of the actually formed structure after printing, compared to the designed area (designed pore size) using image analysis software. As shown in the right graph of FIG. 4B, the printability was the best at 88.5% when a molecular sieve with a mesh diameter of 50 μm was used, and it gradually decreased as the mesh diameter increased, decreasing to 7.8% when a molecular sieve with a mesh diameter of 800 μm was used. In summary, it was confirmed that as the mesh diameter increases, the yield and cell viability also tend to increase, but printability tends to decrease.

In the blending process using a syringe connector connected to a nozzle, a homogeneous tissue strand ink was prepared by repeating the movement in the syringe connector nozzle. At this time, nozzles with diameters of 1.2, 1.4, and 2.4 mm were used, and the yield, cell viability, and printability of the tissue strand ink according to the nozzle diameter were confirmed.

The yield, cell viability, and printability of the tissue strand ink were evaluated in the same manner as described above. As a result, as shown in FIG. 4C, the yield of the tissue strand ink tended to decrease as the nozzle diameter increased (see the left graph of FIG. 4C), and the cell viability was maintained at about 70% or more regardless of the nozzle diameter (see the middle graph of FIG. 4C). Meanwhile, as shown in the right graph of FIG. 4C, the printability was 69.2% at a diameter of 1.2 mm, which was confirmed to be the highest resolution compared to the other groups. Therefore, it can be seen that the printability tends to decrease as the nozzle diameter increases (1.4 mm: 62.8%, 2.4 mm: 54.5%). In summary, it can be confirmed that as the nozzle diameter increases, cell viability tends to increases, but the yield and printability tend to decrease.

### Example 6

### Fabrication of 3D-Printed Artificial Structures Using Tissue Strand Inks Based on Stem Cell/DECM Self-Assembly

The tissue strand ink prepared in the above Example 5 was finally filled into a printing syringe to prepare an artificial tissue having a 3D shape (FIG. 5A). In order to fabricate a structure of a desired shape by applying the tissue strand ink to a 3D printer, the printing conditions were set as described below.

Since the stem cell/DECM self-assembly-based tissue strand ink prepared in Example 5 contained living cells and ECM, a nozzle size of 200 μm or more, an air pressure of less than 80 kPa, and a printing speed of 1 mm/see were used to maximize cell viability during printing.

As a result, as shown in FIG. 5A, it was possible to fabricate not only a fine cross-shaped structure with a size of 500 μm, but also a large structure with a size of 1 cm or more, and as shown in FIG. 5B, it was confirmed that a cell viability of 80% or more was maintained even after printing.

### Example 7

Biochemical Characteristics of Tissue-Specific Artificial Tissue Printed with Stem Cell/DECM Self-Assembly-Based Tissue Strand Ink
7-1. Visual Observation of Artificial Tissue Printed with Tissue-Specific Tissue Strand Ink

Visual observation was performed to confirm whether the artificial tissue printed with the stem cell/DECM self-assembly-based tissue ink was printed according to the set size.

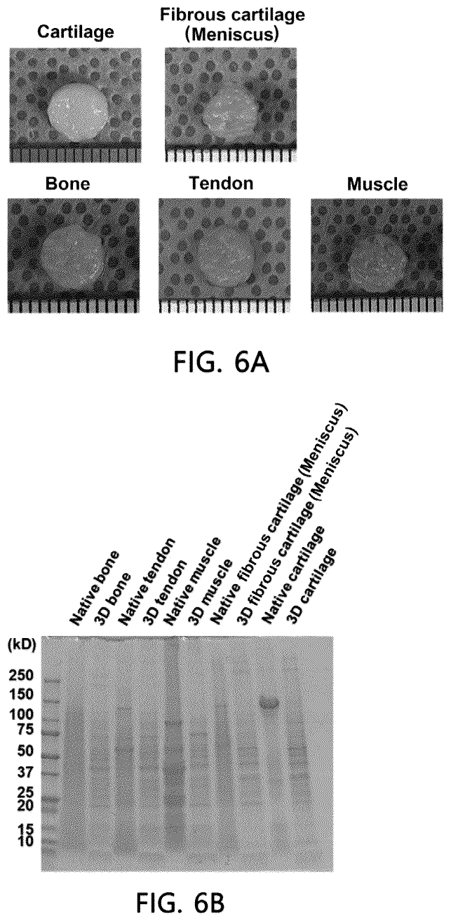

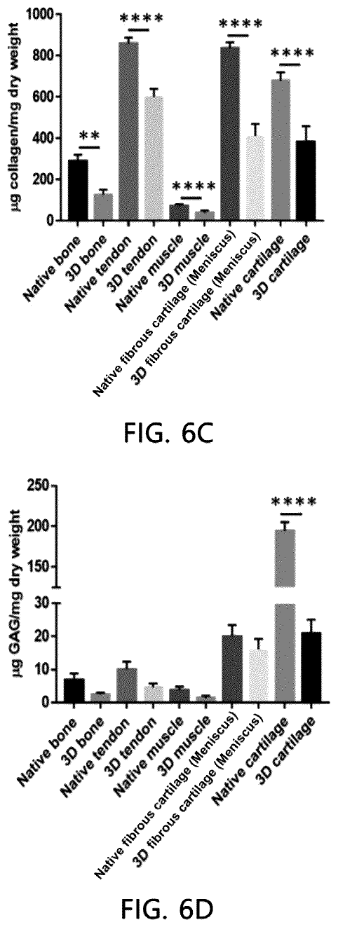

As shown in FIG. 6A, a disk-shaped structure with a diameter of 5 mm was successfully fabricated as designed.

7-2. Analysis of Biochemical Characteristics of Artificial Tissue Printed with Tissue-Specific Tissue Strand Ink

A sodium dodecyl sulfate-polyacrylamide gel electrophoresis (SDS-PAGE) analysis was performed to analyze the protein cargo profile of the artificial tissue printed with each tissue-specific tissue strand ink.

As a result, as shown in FIG. 6B, the expression of major protein bands found in the parent tissue was also found in the printed artificial tissue, confirming that the artificial tissue had biochemical characteristics similar to those of the parent tissue.

In addition, the degree of implementation of parent tissue in the artificial tissue was evaluated through quantitative evaluation of collagen and glycosaminoglycan (GAG), which are major components of the musculoskeletal tissue ECM. As shown in FIG. 6C, it was confirmed that the collagen content of the printed artificial tissue was 60% to 170% of the parent tissue (compared to parent tissue, cartilage: 172%, meniscus: 124%, bone: 96%, ligament: 100%, muscle: 61%). As shown in FIG. 6B, in the case of sGAG, it was also confirmed that the collagen content was 30% to 100% of the parent tissue (compared to parent tissue, cartilage: 76%, meniscus: 46%, bone: 32%, ligament: 103%, muscle: 57%).

### Example 8

### Tissue Differentiation Analysis of Tissue-Specific Artificial Tissue Printed With Stem Cell/DECM Self-Assembly-Based Tissue Strand Ink

To evaluate whether the artificial tissue had biological characteristics similar to those of the parent tissue after differentiation induction, the artificial tissue was cultured in a differentiation medium for four weeks after printing, and then biochemical and histological analyses of the structures were performed.

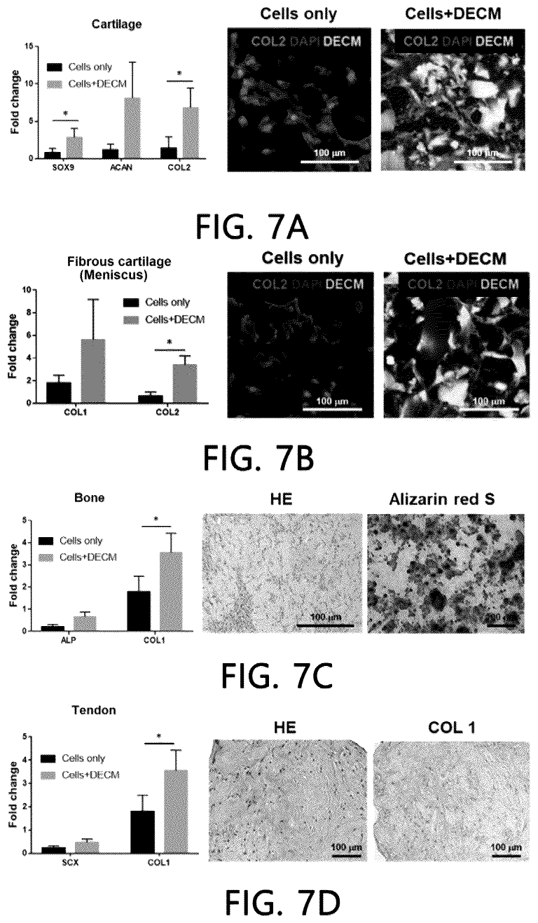

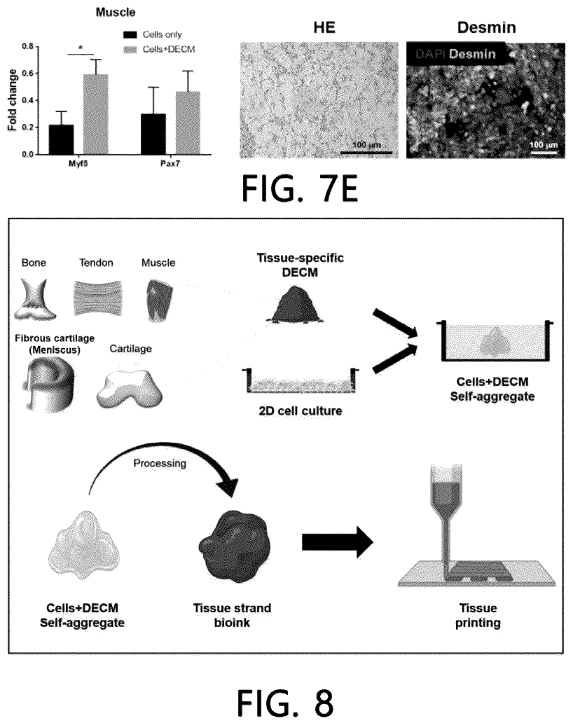

As a result of the RT-qPCR of the artificial tissue prepared with cartilage tissue-derived DECM powder, as shown in FIG. 7A, the expression of cartilage-specific markers SOX9 and COL2 was significantly increased compared to the control group prepared by using only cells. In addition, as a result of immunofluorescence staining, it was confirmed that the expression of collagen type 2, marked in red, increased compared to the control group.

As a result of the RT-qPCR of the artificial tissue prepared using the fibrocartilage DECM powder, as shown in FIG. 7B, the gene expression of type II collagen, which is abundant in fibrocartilage, was significantly increased compared to the control group prepared by using only cells, and the immunofluorescence staining results also confirmed that the expression of proteins increased.

As a result of the RT-qPCR of the artificial tissue prepared using bone tissue-derived DECM powder, as shown in FIG. 7C, it was confirmed that the gene expression of type 1 collagen, a major component of bone tissue, was significantly increased compared to the control group prepared by using only cells, and alkaline phosphatase (ALP) was also increased. As a result of a histological analysis using H&E staining, it was confirmed that the DECM powder and stem cells were homogeneously distributed to form artificial tissue, and it was confirmed that the expression of alizarin red, which indicates calcium accumulation, increased compared to the control group.

As a result of the RT-qPCR of the artificial tissue prepared using ligament-derived DECM powder, as shown in FIG. 7D, it was confirmed that the expression of type 1 collagen, which is a major ECM component of ligaments, and the SCX gene, was significantly increased compared to the control group prepared using only cells. As a result of observing the inside of the artificial tissue through H&E staining, it was confirmed that the DECM powder and cells were homogeneously distributed to form a single artificial tissue, and the evaluation using immunochemical staining confirmed that type 1 collagen was accumulated inside the artificial tissue.

Finally, as a result of the RT-qPCR of the artificial tissue prepared using muscle DECM powder, as shown in FIG. 7E, it was confirmed that the expression of MYF5, a muscle-specific protein, was significantly increased compared to the control group prepared using only cells. As a result of analyzing by immunofluorescence staining, it was confirmed that the expression of desmin protein, a muscle-specific protein, was increased compared to the control group.

From the above description, those skilled in the art to which the present invention pertains will be able to understand that the present invention may be implemented in other specific forms without changing its technical spirit or essential features. In this regard, it should be understood that the above-described embodiments are illustrative in all respects and not restrictive. The scope of the present invention should be construed as including all changes or modifications derived from the meaning and scope of the claims to be described below and equivalent concepts thereof, rather than the above detailed description.

The national research and development project that supported this invention is as follows.
[Project Identification Number] 1711187902
[Project Number] 2023R1A2C100720011
[Ministry] Ministry of Science and ICT
[Project Management (Specialized) Organization] National Research Foundation of Korea
[Name of Research Program] (Type 1-1) Mid-Career Research)
[Research Project] Development of Clinically Applicable Next-generation Artificial Tissue Injection-Type Cartilage Tissue Strand Bioink for Replacing Existing Cell-Biomaterial Mixture Bioink
[Contribution Rate] 1/1
[Name of Project Executing Organization] Ajou University Industry-Academic Cooperation Foundation
[Research Period] Mar. 1, 2023 to Feb. 28, 2027

1. A method for preparing cell-decellularized extracellular matrix (DECM) self-assembly-based 3D printed artificial tissue, comprising:
(a) decellularizing and powdering a tissue-derived extracellular matrix (ECM) to prepare DECM powder;
(b) forming a cell-DECM self-assembly by adding the DECM powder to a culture medium containing cells and then culturing the same;
(c) preparing a tissue strand ink by homogenizing the cell-DECM self-assembly; and
(d) preparing 3D printed artificial tissue by applying the homogenized tissue strand ink to a 3D printing device.

2. The method of claim 1, wherein the tissue of Step (a) is bone, ligament, muscle, fibrocartilage, or cartilage.

3. The method of claim 1, wherein the cells in Step (b) are stem cells.

4. The method of claim 3, wherein the stem cells are one or more selected from the group consisting of mesenchymal stem cells, embryonic stem cells, and induced pluripotent stem cells.

5. The method of claim 1, wherein the DECM powder in Step (b) is added at a concentration of 0.05 to 3 mg/ml.

6. The method of claim 1, wherein the cell-DECM self-assembly in Step (b) is formed in vitro.

7. The method of claim 1, wherein the cell-DECM powder self-assembly in Step (b) is formed by inducing cell proliferation or cell differentiation.

8. The method of claim 1, further comprising: adding a solubilized DECM solution in Step (b).

9. The method of claim 8, wherein the solubilized DECM solution is added at a concentration of 50 to 500 μg/ml.

10. The method of claim 1, wherein Step (b) is culturing for two to nine days after the cells and the DECM powder begin to fuse.

11. The method of claim 1, wherein the homogenizing of the cell-DECM self-assembly in Step (c) is blending by passing the cell-DECM self-assembly obtained after Step (b) through a molecular sieve or through a syringe connector connected to a nozzle.

12. The method of claim 11, wherein the mesh diameter of the molecular sieve is 50 to 800 μm, and the diameter of the nozzle connected to the syringe connector is 1.0 to 3.0 mm.

13. The method of claim 1, wherein the tissue strand ink prepared in Step (d) is injected into a syringe for 3D printing to perform 3D printing with a nozzle size of 200 μm or more under an air pressure of 20 to less than 150 kPa at a printing speed of 0.1 to 3 mm/sec.

14. Cell-decellularized extracellular matrix (DECM) self-assembly-based 3D printed artificial tissue prepared by the method of claim 1.

15. The artificial tissue of claim 14, wherein the artificial tissue exhibits biochemical characteristics of original tissue.

16. A cell-decellularized extracellular matrix (DECM) self-assembly-based 3D printed artificial organ prepared by the method of claim 1.
